(19) United States
(12) Patent Application Publication
(10) Pub. No.: US 2019/0309433 A1
(43) Pub. Date: Oct. 10, 2019

(54) Electro-Deposited Conducting Polymers For The Realization Of Solid-State Reference Electrodes For Use In Intracutaneous And Subcutaneous Analyte-Selective Sensors

(71) Applicants: Sirilak Sattayasamitsathit, San Diego, CA (US); Joshua Windmiller, San Diego, CA (US); Jared Tangney, San Diego, CA (US); Biolinq, Inc., San Diego, CA (US)

(72) Inventors: Sirilak Sattayasamitsathit, San Diego, CA (US); Joshua Windmiller, Del Mar, CA (US); Jared Tangney, Encinitas, CA (US)

(73) Assignee: Biolinq, Inc., San Diego, CA (US)

(21) Appl. No.: 16/334,022

(22) PCT Filed: Oct. 5, 2017
(86) PCT No.: PCT/US17/55314

§ 371 Date: Mar. 17, 2019

Related U.S. Application Data
(60) Provisional application No. 62/406,389, filed on Oct. 10, 2016.

(51) Int. Cl. C25D 9/02; A61B 5/1486; A61B 5/1473; A61B 5/145

(52) U.S. Cl. CPC C25D 9/02; A61B 5/14865; A61B 5/14546; A61B 5/14507; A61B 5/1473

(57) ABSTRACT

A method (300) for constructing a solid-state reference electrode for use in an analyte-selective intracutaneously- or subcutaneously-implanted electrochemical cell is disclosed herein. The method (300) includes immersing a metallic electrode in a solution comprising a monomer precursor. The method (300) also includes applying a fixed or time-varying electrical potential or current to the metallic electrode, thereby serving to simultaneously electro-polymerize the monomer precursor and electro-deposit a conducting polymer, synthesized from the electro-polymerization of the monomer precursor, onto the said metallic electrode surface.

### CROSS REFERENCES TO RELATED APPLICATIONS

The Present application is the United States National Stage of Patent Cooperation Treaty Application Number PCT/US2017/055314, filed on Oct. 5, 2017, which claims priority to U.S. Provisional patent Application No. 62/406,389, filed on Oct. 10, 2016, each of which is hereby incorporated by reference in its entirety.

### STATEMENT REGARDING FEDERALLY SPONSORED RESEARCH OR DEVELOPMENT

Not Applicable

### BACKGROUND OF THE INVENTION

### Field of the Invention

The present invention generally relates to sensors.

### Description of the Related Art

Prior art solutions have been concerned with the creation of quasi- or pseudo-reference electrodes—essentially metallic wires lacking a junction or internal filling solution of any sort; in certain circumstances, these metallic wires are coated with a layer of metal salt, such as a silver wire functionalized on the surface with a layer of silver chloride. Such electrodes fail to maintain a constant potential, albeit vary predictably with known conditions. Under such circumstances, the potential can be calculated and the electrode can be employed as a reference electrode. Such quasi- or pseudo-reference electrodes operate over a limited range of conditions, such as pH, ionic strength, or temperature; exceeding these boundaries and electrode behavior becomes less predictable. With regards to micro-fabricated chip-scale sensors often employed for microfluidic applications, lithographically-defined metal electrodes are utilized as a proxy for the said wires, albeit such devices suffer from the same shortcomings.

Yong Suk Choi et al., U.S. Pat. No. 6,793,789 for a Reference electrode with a polymeric reference electrode membrane discloses a polymeric reference electrode membrane comprising (a) one selected from a porous polymer or a hydrophilic plasticizer; (b) a lipophilic polymer; and optionally an adhesion-enhancing material. A reference electrode equipped with the polymeric reference electrode membrane can be shortened the preconditioning time, and extended lifetime for storage and use owing to excellent adhesion, and showed reproducibility and good yield. So, a miniaturized multi-potentiometric sensor can be fabricated comprising a solid-state reference electrode of the present invention and a set of ion-selective electrodes, thus being useful in the potentiometric fields, including clinical, environmental, food and industrial analysis.

Kinlen et al., U.S. Pat. No. 4,908,117 for a Solid state reference electrode discloses a solid state reference electrode comprising a metal/metal salt electrode, an immobilized electrolyte in contact with the metal salt, and a perfluorocarbon copolymer coating on the immobilized electrolyte to prevent migration of the electrolyte away from the electrode.

Zick et al., U.S. Pat. No. 4,450,842 for a Solid state reference electrode discloses in the reference electrode construction disclosed herein, silver and silver chloride particles are incorporated in a mixture including a fusible glass frit. After being printed on a suitable substrate, the mixture is fired to bond the silver/silver chloride composite into a conductive film on the surface of the substrate.

The prior art fails to disclose a viable solid-state reference electrode in micron-scale geometries.

### BRIEF SUMMARY OF THE INVENTION

The reference electrode serves as a requisite constituent of an electrochemical cell and is necessary to impart stable and controlled electrochemical reactions. Although such electrodes are commonplace in benchtop experiments, their architecture precludes these devices from integration in miniaturized platforms. Often, when attempts are made to integrate reference electrodes in micron-scale geometries, such as within semiconductor chips, the stability of the electrode is compromised. Indeed, the creation of a stable solid-state reference electrode has posed a formidable challenge to those aiming to create intracutaneously- and subcutaneously-implanted electrochemical sensors for the quantification of circulating analytes in physiological fluids.

The technology described herein relates to implantable, analyte-selective electrochemical sensors and methods for producing the same.

One aspect of the present invention is a method for constructing a solid-state reference electrode for use in an analyte-selective intracutaneously- or subcutaneously-implanted electrochemical cell. The method includes immersing a metallic electrode in a solution comprising a monomer precursor. The method also includes applying a fixed or time-varying electrical potential or current to the metallic electrode, thereby serving to simultaneously electro-polymerize the monomer precursor and electro-deposit a conducting polymer, synthesized from the electro-polymerization of said monomer precursor, onto the said metallic electrode surface.

Another aspect of the present invention is an analyte-selective intracutaneously- or subcutaneously-implanted electrochemical cell device. The device comprises a contingent of one or multiple metallic electrodes, and a layer of electro-deposited conducting polymer on said metallic electrode contingent to form a solid-state reference electrode with a stable electrode potential. The electrochemical cell device refers electrochemical measurements at a working electrode against said solid-state reference electrode.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 illustrates a conventional reference electrode with the major components of the device indicated.

FIG. 2 illustrates an optical micrograph delineating two bare platinum electrodes (top row) and two conducting polymer-coated platinum electrodes (bottom row) on a silicon die.

FIG. 3 illustrates a block/process flow diagram illustrating the major constituents of the solid-state reference electrode manufacturing process.

FIG. 4 illustrates a diagrammatic representation of the prior art delineating the major functional components required for the construction of a conventional liquid junction reference electrode.

FIG. 5 illustrates an electro-polymerization of the pyrrole monomer precursor (left) in the presence of an oxidizing electrical potential to form the conducting polymer poly(pyrrole) (right).

FIG. 6 illustrates a detailed schematic representation of the electro-polymerization process of poly(pyrrole).

### DETAILED DESCRIPTION OF THE INVENTION

The present invention discloses a method for the synthesis of a viable, stable, solid-state reference electrode with noteworthy analytical merits. Namely, a conducting polymer is electro-deposited onto the surface of a micron- or nano-scale electrode following immersion of said electrode into a solution comprising a precursor compound to the conducting polymer, a monomer, which is simultaneously electro-polymerized to form the conducting polymer during the said electro-deposition process. This method yields a viable, stable, solid-state reference electrode. Optionally, a dopant counter anion or cation is included in the solution to provide for an abundance of available charge carriers to improve electrical conduction properties and facilitate ion exchange when the as-synthesized reference electrode is utilized in physiological fluids containing like ions.

In order to impart high-fidelity sensing over extended durations, electrochemical sensors leverage reference electrodes featuring a metal electrode/liquid/semi-permeable membrane architecture to facilitate the establishment of a stable and thoroughly-characterized electrode potential from which potentials applied or measured at other electrodes in the electrochemical cell are referred. This potential remains insensitive with regards to fluctuations in (1) the chemical composition of the solution in which the reference electrode is immersed and (2) the passage of current through a separate working and counter electrode contingent. Such reference electrodes, which ideally are non-polarizable, are constructed via immersion of a metal wire (often coated with a salt of the metal) into a capillary tube filled with a saturated ionic solution (“internal filling solution”) such that ion-exchange occurs between the metal (or metal salt) and the ionic solution at a precisely-defined potential under redox equilibrium. A semi-permeable membrane, located at the distal end of the capillary tube, facilitates electrical contact between the internal filling solution and the solution in which the reference electrode is immersed. Although this approach is appropriate for benchtop evaluation, the construction of such confined liquid junctions at the micron scale, especially in semiconductor manufacturing embodiments, is not easily achieved using conventional fabrication approaches. The invention disclosed provides for a means for the realization of completely solid-state reference electrodes that obviate the need for said internal filling solutions and semi-permeable membranes employed in the prior art, thereby positioning the innovation for used in micro-fabricated electrochemical cells such as sub- and intra-cutaneous analyte-selective sensors.

The present invention represents an alternative approach facilitating the synthesis of a viable solid-state reference electrode that addresses the shortcomings of the prior art while remaining amenable to highly scalable manufacturing processes. These shortcomings include:

(1) The necessity of implementing an internal filling solution, which maintains thermodynamic redox equilibrium with a metal/metal-salt electrode immersed within, thereby yielding a stable electrical potential: Limitation: (a) An internal filling solution cannot be easily implemented under micro-fabrication embodiments using conventional high-throughput manufacturing processes used in the semiconductor or microelectronics industries. (b) Mitigation: Utilization of a conducting polymer containing an embedded dopant ion to result in the establishment of a stable electrode potential that maintains thermodynamic redox equilibrium with the underlying metal electrode.

(2) The mechanical/hand assembly of conventional reference electrodes, which are multi-component in nature: (a) Limitation: Mechanical or hand assembly are not amenable to micro- or nano-scale electrode dimensions that are typically encountered in micro-fabricated systems. (b) Mitigation: Utilization of an electro-deposition process to deposit a conducting polymer film on a metallic electrode of nearly any geometry. The electro-deposition process is amenable to automated, scalable, and highly parallelized fabrication of highly uniform functional reference electrodes that obviate the need for mechanical/hand assembly, as is often done with conventional reference electrodes.

(3) The realization of micro-fabricated metal-salt electrodes: (a) Limitation: Metal salt electrodes require chemical or electrochemical processing of an elemental metal precursor and cannot be easily synthesized, as is the case with elemental metal electrodes, using micro-manufacturing equipment. (b) Mitigation: The coating of an elemental metallic or metal alloy electrode with a conducting polymer containing an embedded dopant ion exhibits identical redox behavior to conventional metal-salt-coated metal electrodes when immersed in solution.

(4) Semi-permeable membrane junctions, which forms a half-cell: (a) Limitation: Conventional reference electrodes make extensive use of glass frit semi-permeable membranes, which facilitate electrical communication between the internal filling solution within the reference electrode and the surrounding solution in which said reference electrode is immersed. The fabrication of said semi-permeable membranes employing an automated or semiconductor manufacturing process is not easily achieved owing to the inability to process the material using established micro-manufacturing techniques. (b) Mitigation: The implementation of a conducting polymer emulates the semi-permeability of a conventional glass frit membrane. Said conducting polymer enables the electrical communication between the underlying metal electrode and the solution in which the as-fabricated solid-state reference electrode is immersed.

(5) Inability to scale to micron- and sub-micron dimensions: (a) Limitation: Owing to their multi-component design and extensive use of materials that are incompatible with conventional micro-fabrication and semiconductor manufacturing methods, conventional reference electrodes present difficulty when attempting to scale below centimeter-scale dimensions. (b) Mitigation: Employing a fabrication/manufacturing technique and materials that are compatible with the existing semiconductor processing infrastructure ensures that said reference electrodes can be scaled to micron- or sub-micron dimensions and integrated alongside functional electrical circuits, microfluidic systems, and micro-electromechanical systems.

The technology disclosed herein preferably requires the immersion of at least two metallic electrodes (with one selected to serve as the reference electrode) in an aqueous or non-aqueous solution comprising a monomer precursor and, optionally, a dopant ion. A constant or time-varying electrical potential or current is applied to the two metallic electrodes for a specified amount of time or until a specified amount of charge has passed.

Alternatively, the technology disclosed herein preferably requires the immersion of at least two metallic electrodes (with one selected to serve as the counter electrode) and one liquid-junction reference electrode in an aqueous or non-aqueous solution comprising a monomer precursor and, optionally, a dopant ion. A constant or time-varying electrical potential or current is applied to the two metallic electrodes (and referenced against the said reference electrode) for a specified amount of time or until a specified amount of charge has passed.

Two scenarios can occur: (1) Through an oxidative process in which one or more electrons are removed from the monomer precursor molecule, the said monomer cross-links with other monomer units to maintain charge neutrality and forms a conducting polymer chain when used as the anode in the system, otherwise referred to as anodic electro-polymerization. This forms a p-type conducting polymer (i.e. a material in which electron holes serve as the charge carrier). Simultaneously, the conducting polymer is deposited on the electrode surface, via electrostatic interaction, in a process known as anodic electro-deposition. (2) Through a reductive process in which one or more electrons are transferred to the monomer precursor molecule, the monomer cross-links with other monomer units to maintain charge neutrality and forms a conducting polymer chain when used as the cathode in the system, otherwise referred to as cathodic electro-polymerization. This forms an n-type conducting polymer (i.e. a material in which electrons serve as the charge carrier). Simultaneously, the conducting polymer is deposited on the electrode surface, via electrostatic interaction, in a process known as cathodic electro-deposition.

In each of the above-mentioned scenarios, the presence of a monoatomic or polyatomic anion or cation in the solution (containing the monomer) encourages the “doping” process. In the case of inclusion of an anion in the solution, the uptake of said ion during the electro-polymerization process for inclusion in the conducting polymer will result in a conducting polymer that is n-type. Alternatively, in the case of inclusion of a cation in the solution, the uptake of said ion during the electro-polymerization process for inclusion in the conducting polymer results in a conducting polymer that is p-type.

Under each of the above scenarios, the as-electro-deposited conducting polymer also serves a tandem function as a semi-permeable membrane that emulates the same in a conventional reference electrode without requiring an internal filling solution, hence facilitating electrical conduction between the underlying metallic electrode and the solution in which it is immersed.

Metal electrode: A metal surface, of defined geometry and surface morphology, electrically addressed by an external circuit. The metal surface preferably comprises, but is not limited to, one or more of the following elemental metals: chromium, titanium, tin, nickel, copper, silver, gold, platinum, palladium, rhodium, and iridium. Metal alloys (combination of two or more metals or metalloids) may also be employed.

Conducting polymer: An organic polymer able to conduct an electrical current. The conducting polymer is preferably doped with a counter anion(s) or cation(s) to improve electrical conductivity and provide for ion exchange with the solution in which it is immersed (such as physiological fluid—blood, plasma, extracellular fluid, intracellular fluid, interstitial fluid, cerebrospinal fluid, for example). The conducting polymer is electro-deposited onto the surface of the metal electrode to form the reference electrode.

A method for generating the electrode begins with immersion of at least two metal electrodes in a solution comprising a monomer precursor. The at least two metal electrodes are connected to a voltage or current source, which facilitates the application of a potential or the sourcing of a current to/through the electrode pair and the formation of an electrochemical cell; one of the electrodes (selected as the anode or cathode, depending on the desired redox reaction) will be functionalized as the reference electrode.

Next, application of a fixed or time-varying electrical potential or current from a voltage source or a current source to the metallic electrode contingent serves to simultaneously electro-polymerize the monomer precursor and electro-deposit a conducting polymer, synthesized from the electro-polymerization of the monomer precursor, onto the metallic electrode surface.

The inputs of the invention include a metal electrode, a solution comprising monomer precursor, a dopant ion and a constant or time-varying electrical potential or current.

In a preferred embodiment, the metal electrode has a metal, metalloid, or metal alloy surface, of defined geometry and surface morphology, electrically addressed by an external circuit. The metal surface preferably comprises, but is not limited to, one or more of the following metals: chromium, titanium, tin, nickel, copper, silver, gold, platinum, palladium, rhodium, and iridium, or alloy of two or more elemental metals.

In a preferred embodiment, the solution comprising the monomer precursor comprises, but is not limited to, one of the following monomeric compounds: aniline, acetylene, phenylene, phenylene-diamine, phenylene-vinylene, phenylene-sulfide, pyrrole, thiophene, and 3,4-ethylenedioxythiophene.

In a preferred embodiment, the dopant ion (optional, in solution along with monomer) is employed as a charge carrier to provide improved electrical conduction within the conducting polymer matrix. The dopant ion also facilitates ion-exchange with like-ions in the sensing medium, such as physiological fluid. The dopant ion preferably comprises, but are not limited to, anions including chloride, sulfide, bicarbonate, sulfate, phosphate, and nitrate, or cations including hydrogen, lithium, sodium, potassium, calcium, and magnesium.

In a preferred embodiment, the constant or time-varying electrical potential or current is employed to instigate the simultaneous electro-polymerization of the monomer precursor and electro-deposition of the resultant conducting polymer onto the metal electrode. The constant or time-varying electrical potential or current is preferably applied using one of amperometry, voltammetry, and coulometry.

The output is a solid-state reference electrode, which comprises the metal electrode coated with the electro-polymerized/electro-deposited conducting polymer, optionally containing a dopant anion or cation, and it is used in conjunction with one or more separate electrodes to form an electrochemical cell.

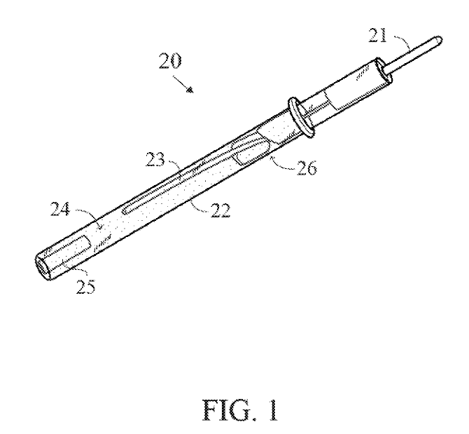

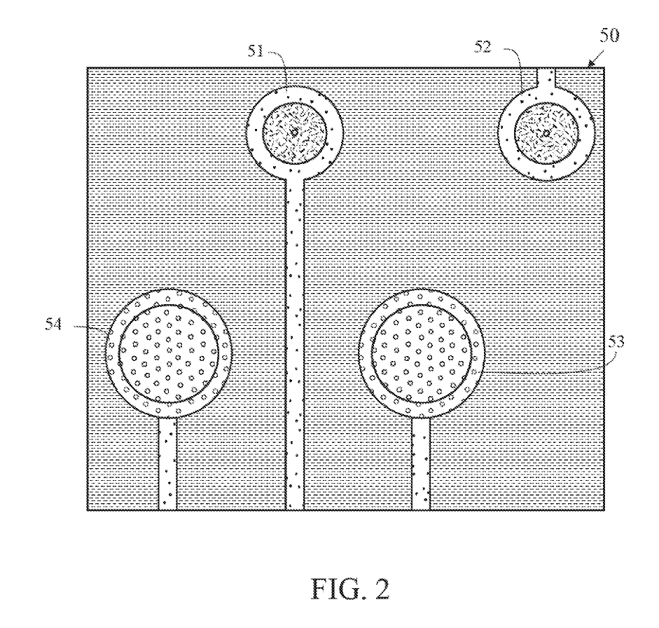

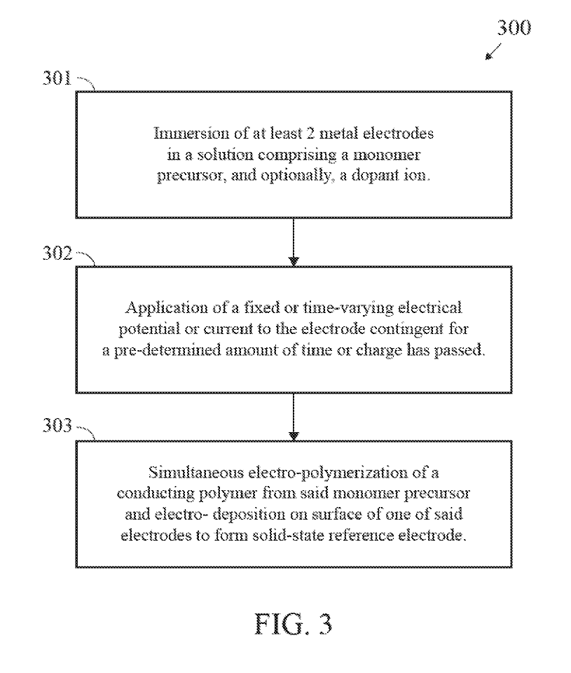

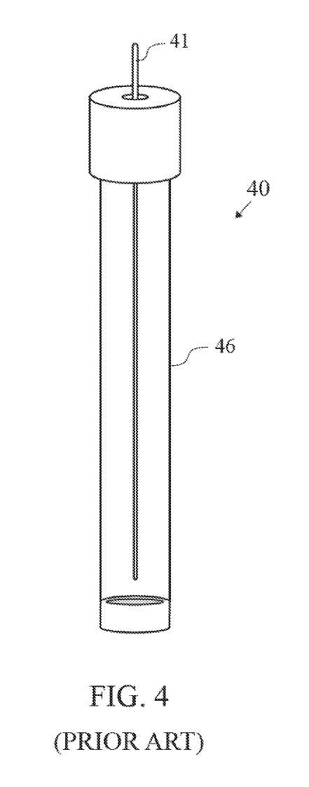

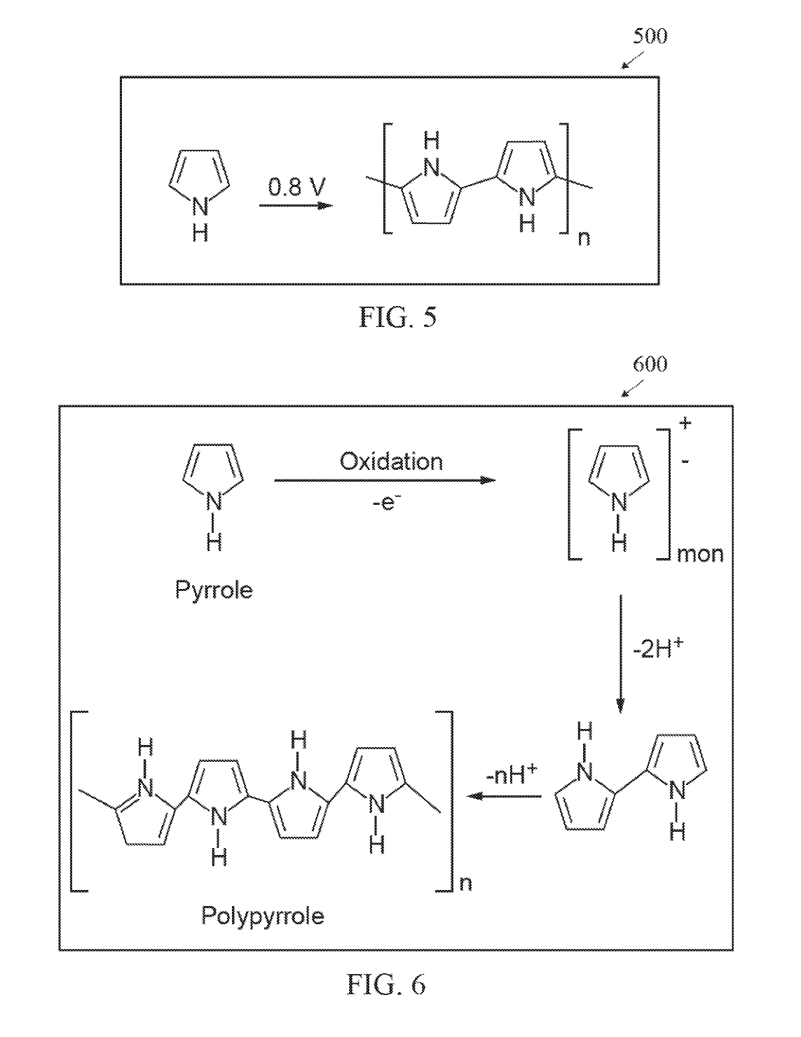

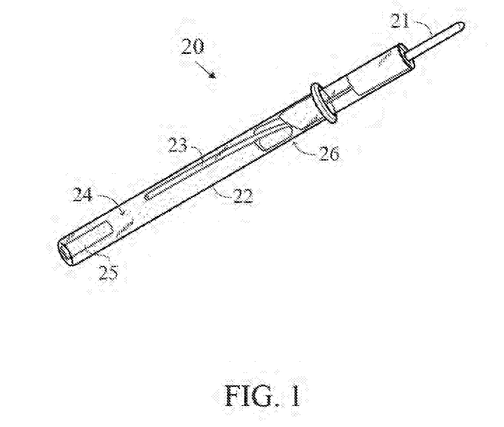

FIG. 1 illustrates a conventional reference electrode with major components of the device 20 including an electrode connector 21, a glass capillary 22, a metal wire 23, an internal ionic filling solution 24, a semi-permeable membrane 25 and a body 26. In this example, a silver/silver-chloride reference electrode, comprising a silver-chloride-coated silver wire immersed in a 1M potassium chloride solution used in conjunction with a Vycor® semi-porous glass frit membrane, is shown.

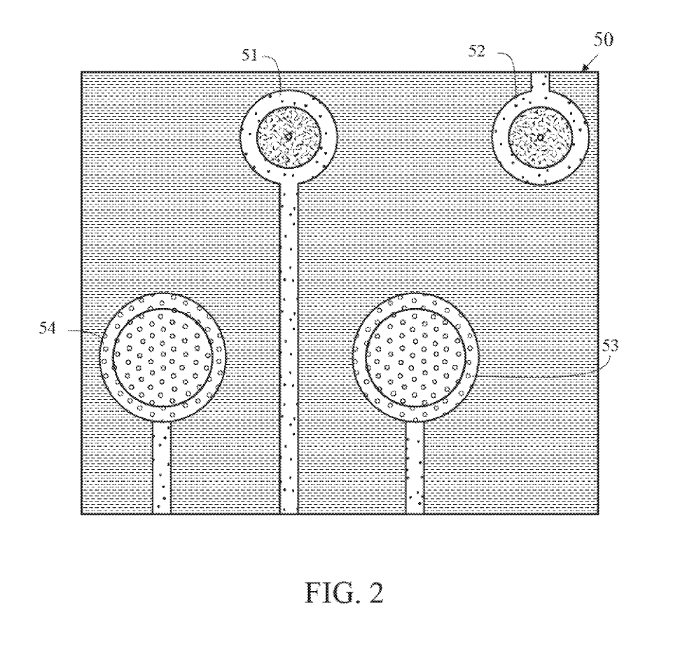

FIG. 2 illustrates an optical micrograph 50 delineating two bare platinum electrodes 51 and 52 (top row) and two conducting polymer-coated platinum electrodes 53 and 54 (bottom row) on a silicon die; both conducting polymer-coated platinum electrodes are used as solid-state, chip-scale reference electrodes. The diameter of each electrode is preferably 135 μm.

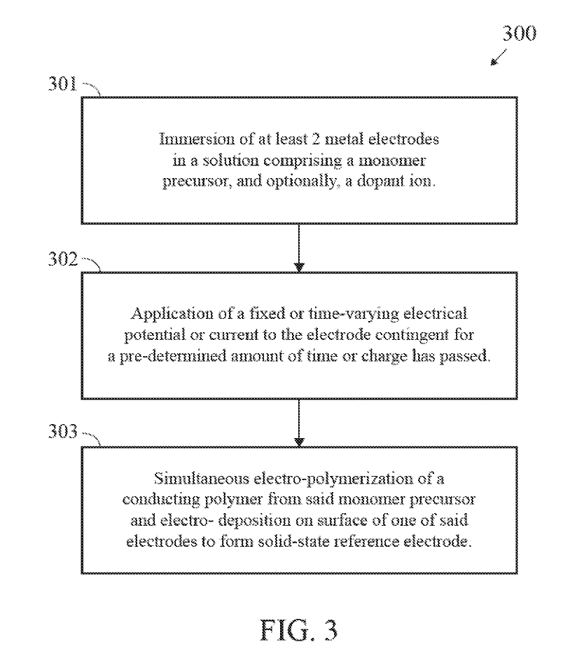

FIG. 3 illustrates a block/process flow diagram illustrating the major constituents of the solid-state reference electrode manufacturing process. A method 300 for constructing a solid-state reference electrode for use in an analyte-selective intracutaneously- or subcutaneously-implanted electrochemical cell is provided. At block 301 a metallic electrode is immersed in a solution comprising a monomer precursor. At block 302, a fixed or time-varying electrical potential or current is applied to the metallic electrode, thereby serving to simultaneously electro-polymerize the monomer precursor and electro-deposit a conducting polymer, synthesized from the electro-polymerization of said monomer precursor, onto the said metallic electrode surface. At block 303, the simultaneous electro-polymerization of a conducting polymer and the electro-deposition on the surface of at least one electrode occurs.

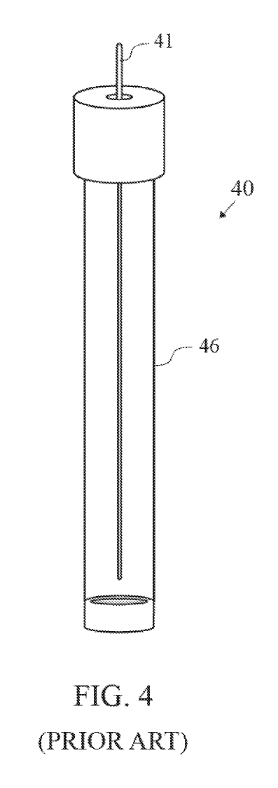

FIG. 4 illustrates a diagrammatic representation of the prior art delineating the major functional components required for the construction of a conventional liquid junction reference electrode 40 with a body 46 and an electrode connector 41.

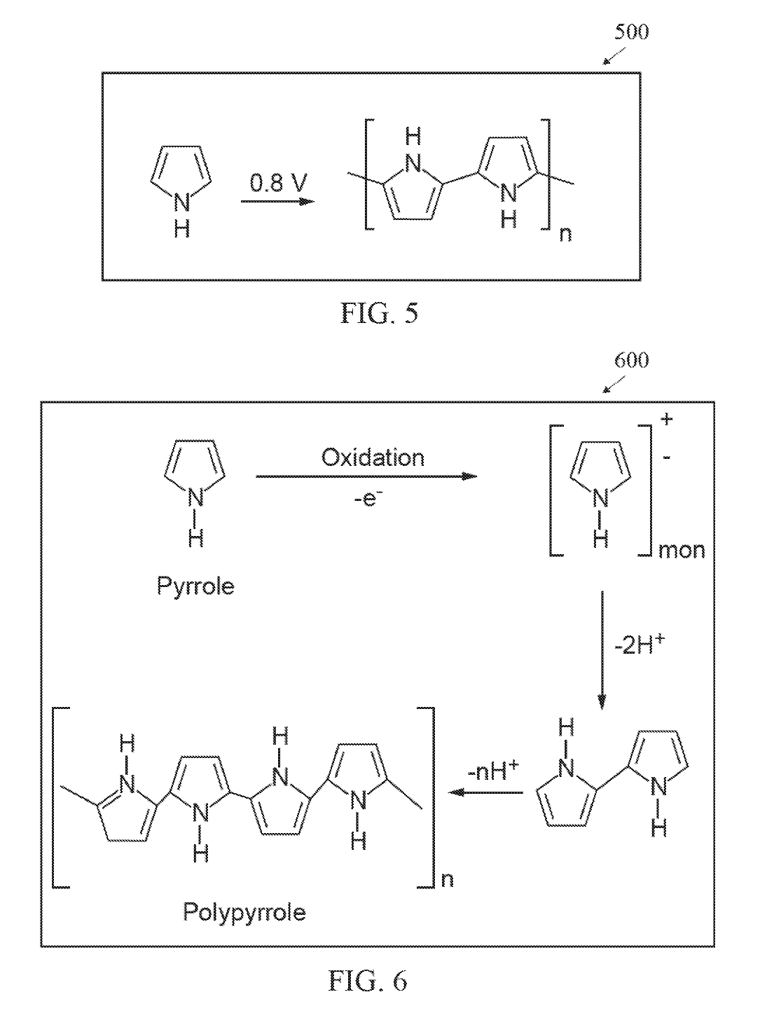

FIG. 5 illustrates an electro-polymerization process 500 of the pyrrole monomer precursor (left) in the presence of an oxidizing electrical potential to form the conducting polymer poly(pyrrole) (right).

FIG. 6 illustrates a further detailed schematic representation of the electro-polymerization process 600 of poly(pyrrole). Following the arrows: The pyrrole monomer (left, top); upon application of an oxidation potential, an electron is removed from the monomer, leading to the formation of a radical oxidized monomer carrying positive charge (right, top); with continued application of an oxidation potential of sufficient magnitude, a hydrogen bond is formed between two adjacent pyrrole monomers to maintain charge-neutrality (right, bottom); further application of said oxidation potential results in the formation of a poly(pyrrole) conducting polymer chain. The de-localized electron holes formed during the electro-polymerization process facilitate electrical conductivity (i.e. p-type charge carrier).

One embodiment is an analyte-selective intracutaneously- or subcutaneously-implanted electrochemical cell device. The device comprises a contingent of one or multiple metallic electrodes, and a layer of electro-deposited conducting polymer on said metallic electrode contingent to form a solid-state reference electrode with a stable electrode potential. The electrochemical cell device refers electrochemical measurements at a working electrode against said solid-state reference electrode.

The metallic electrode is selected from the group consisting of chromium, titanium, tin, nickel, copper, silver, gold, platinum, palladium, rhodium, and iridium.

The conducting polymer preferably comprises at least one of poly(aniline), poly(acetylene), poly(phenylene), poly(phenylene-diamine), poly(phenylene-vinylene), poly(phenylene-sulfide), poly(pyrrole), poly(thiophene), poly(3,4-ethylenedioxythiophene), or the derivatives of any of the aforementioned polymers. The conducting polymer also preferably contains a counter anion or cation to serve as the charge carrier or dopant. The monoatomic counter cation preferably includes hydrogen, lithium, sodium, potassium, calcium, or magnesium. The polyatomic counter cation preferably includes ammonium. The monoatomic counter anion preferably includes fluoride, chloride, sulfide, bromide, or iodide. The polyatomic counter anion preferably includes sulfate, bicarbonate, phosphate, nitrate, hydroxide, peroxide, acetate, carbonate, or polystyrene sulfonate. Alternatively, said conducting polymer contains an aggregate of two or more counter anions or two or more counter cations to enhance the conductivity or charge transfer properties of said conducting polymer.

Another embodiment is the incorporation of a substance encouraging proteolytic activity, anti-oxidant activity, or inhibiting microbial activity in said monomer precursor solution, along with any relevant dopant ions, in order to reduce the likelihood of biofouling, oxidative stress, or infection, respectively, when said solid-state reference electrode is implanted in a living biological system. Said proteolytic substance can comprise a protease enzyme. Said anti-oxidant substance can comprise a catalase enzyme, a superoxide dismutase enzyme, a glutathione reductase enzyme, a glutathione peroxidase enzyme, or a peroxidase enzyme. Said anti-microbial substance can comprise a lysozyme enzyme or antibiotic compound.

Another embodiment is the application of a high electrical potential or current or the application of said electrical potential or current for a sufficiently extended period of time to encourage the over-oxidation or over-reduction of said as-deposited conducting polymer. This process serves to enhance the conductivity of said conducting polymer as well as its charge-transfer properties. Said over-oxidation or over-reduction process can occur simultaneously during the electro-polymerization/electro-deposition process or following said electro-polymerization/electro-deposition process, either in the solution containing the monomer precursor (along with any dopant ions) or in a new solution entirely. Said new solution can comprise any aqueous or non-aqueous solution.

Another embodiment is the application of one or more conducting polymer layers on the surface of said conducting polymer using a process identical to that mentioned [above]. Said one or more conducting polymer layers can either contain a single counter anion, single counter cation, plurality of counter anions, plurality of counter cations, or no ions whatsoever. Said one or more conducting polymer layers can either comprise the same conducting polymer substance as the first layer or comprise a different conducting polymer substance entirely. Said one or more conducting polymer layers can either be over-oxidized, over-reduced, or be unperturbed.

Another embodiment is an electrochemical cell device comprising at least one metallic electrode, and a layer of a conducting polymer electro-deposited on the metallic electrode to form a solid-state reference electrode with a stable electrode potential. The electrochemical cell device refers electrochemical measurements at a working electrode against the solid-state reference electrode. The metallic electrode preferably possesses a two-dimensional geometric surface. The metallic electrode alternatively possesses a three-dimensional geometric surface. The metallic electrode is preferably structured to possess a shape including at least one of cylindrical, conical, circular, triangular, pyramidal, quadrilateral, or polygonal. The spatial extent of the two most distant features of the metallic electrode is preferably between 2 and 4000 micrometers.

Yet another embodiment is a method for constructing a solid-state reference electrode for use in an analyte-selective intracutaneously- or subcutaneously-implanted electrochemical cell. The method includes immersing a metallic electrode in a solution comprising a monomer precursor. The method also includes applying a fixed or time-varying electrical potential or current to the metallic electrode, thereby serving to simultaneously electro-polymerize the monomer precursor and electro-deposit a conducting polymer, synthesized from the electro-polymerization of said monomer precursor, onto the said metallic electrode surface. The monomer precursor is present in concentration preferably between 0.001 and 3.000 moles per liter (M).

The electrical potential resides in a range preferably between −1.2 and +1.2 V versus an internal or external reference electrode. The electrical current resides in a range preferably between +/−1×10(−12) and +/−1 Ampere.

The metallic electrode has a surface area between 1×(−9) and 1×(−3)m\hat{ }2 [deposited conducting polymer will have same area]. The conducting polymer is preferably deposited to a thickness between 1×(−9) and 1×(−4)m on the underlying metallic electrode.

The conducting polymer is deposited in a geometry that is identical to the geometry embodied by the underlying metallic electrode.

The counter anion or counter cation is present in concentration between 1×10\hat{ }(−4) and 3 moles per liter (M).

Wang et al., U.S. Patent Publication Number 20140336487 for a Microneedle Arrays For Biosensing And Drug Delivery is hereby incorporated by reference in its entirety.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

1. A method for constructing a solid-state reference electrode for use in an analyte-selective intracutaneously- or subcutaneously-implanted electrochemical cell, said method comprising:
immersing a metallic electrode in a solution comprising a monomer precursor;
applying a fixed or time-varying electrical potential or current to the metallic electrode, thereby serving to simultaneously electro-polymerize the monomer precursor and electro-deposit a conducting polymer, synthesized from the electro-polymerization of said monomer precursor, onto the said metallic electrode surface;
wherein the conducting polymer comprises at least one of poly(aniline), poly(acetylene), poly(phenylene), poly(phenylenediaminie), poly(phenylene-vinylene), poly(phenylene-sulfide), poly(thiophene) and poly(3,4-ethylenedioxythiophene).

2. The method of claim 1, wherein metallic electrode includes chromium, titanium, tin, nickel, copper, silver, gold, platinum, palladium, rhodium, and iridium.

3. (canceled)

4. The method of claim 1, wherein said monomer precursor includes at least one of aniline, acetylene, phenylene, phenylenediamine, phenylene-vinylene, phenylene-sulfide, pyrrole, thiophene, and 3,4-ethylenedioxythiophene.

5. The method of claim 1, further comprising forming two or more layers of the conducting polymer.

6. The method of claim 1, wherein said solution contains a counter anion or cation to serve as the charge carrier or dopant within the conducting polymer.

7. The method of claim 6, wherein said counter anion includes at least one of chloride, sulfate, phosphate, and nitrate.

8. The method of claim 6, wherein said counter cation includes at least one of hydrogen, lithium, sodium, potassium, calcium, and magnesium.

9. The method of claim 1, wherein said fixed or time-varying electrical potential or current is applied using one of amperometry, voltammetry, and coulometry.

10. The method of claim 1, wherein said fixed or time-varying electrical potential or current is applied to encourage the formation of a radical anion or cation, thereby yielding charge carriers within the electro-deposited conducting polymer.

11. The method of claim 1, wherein said fixed or time-varying electrical potential or current is applied to reduce or oxidize the monomer precursor, thereby giving rise to charge carriers within the electro-deposited conducting polymer.

12. An analyte-selective intracutaneously- or subcutaneously-implanted electrochemical cell device, said device comprising:
a contingent of one or multiple metallic electrodes; and
a layer of electro-deposited conducting polymer on said metallic electrode contingent to form a solid-state reference electrode with a stable electrode potential;
wherein said electrochemical cell device refers electrochemical measurements at a working electrode against said solid-state reference electrode;
wherein the conducting polymer comprises at least one of poly(aniline), poly(acetylene), poly(phenylene), poly(phenylenediaminie), poly(phenylene-vinylene), poly(phenylene-sulfide), poly(thiophene) and poly(3,4-ethylenedioxythiophene).

13. The device of claim 12, wherein metallic electrode includes at least one of chromium, titanium, tin, nickel, copper, silver, gold, platinum, palladium, rhodium, and iridium.

14. (canceled)

15. The device of claim 12, wherein said conducting polymer contains a counter anion or cation to serve as the charge carrier or dopant, wherein said counter anion includes at least one of chloride, sulfate, phosphate and nitrate, and wherein the counter cation includes at least one of hydrogen, lithium, sodium, potassium, calcium and magnesium.

16-28. (canceled)

29. A method for constructing a solid-state reference electrode, the method comprising:
immersing a metallic electrode in a solution comprising a monomer precursor;
applying a time varying electrical potential to the metallic electrode, to simultaneously electro-polymerize the monomer precursor and electro-deposit a conducting polymer onto a surface of the metallic electrode to form a solid state reference electrode, wherein the conducting polymer is synthesized from the electro-polymerization of the monomer precursor;
wherein the solution comprises a counter cation as a charge carrier and the counter cation is one of hydrogen, lithium, sodium, potassium, calcium, and magnesium;
wherein the monomer precursor comprises at least one of aniline, acetylene, phenylene, phenylenediamine, phenylene-vinylene, phenylene-sulfide, thiophene, and 3,4-ethylenedioxythiophene;
wherein the conducting polymer comprises at least one of poly(aniline), poly(acetylene), poly(phenylene), poly(phenylenediaminie), poly(phenylene-vinylene), poly(phenylene-sulfide), poly(thiophene), and poly(3,4-ethylenedioxythiophene).

30. The method according to claim 29 wherein the metallic electrode is composed of one of chromium, titanium, tin, nickel, copper, silver, gold, platinum, palladium, rhodium, and iridium.

31-32. (canceled)

33. The method according to claim 32 further comprising forming two or more layers of the conducting polymer.

34-36. (canceled)

37. The method according to claim 29 wherein the time varying electrical potential is applied using one of amperometry and coulometry.

38. The method according to claim 29 wherein the time varying electrical potential is applied to encourage the formation of a radical anion or cation, to yield charge carriers within the electro-deposited conducting polymer.

39. The method according to claim 29 wherein the time varying electrical potential is applied to reduce or oxidize the monomer precursor, to generate charge carriers within the electro-deposited conducting polymer.

40-46. (canceled)
